(19) United States
(12) Patent Application Publication
(10) Pub. No.: US 2016/0040208 A1
(43) Pub. Date: Feb. 11, 2016

(54) METHOD FOR STABILISING OXIDATION-SENSITIVE METABOLITES PRODUCED BY MICROALGAE OF THE CHLORELLA GENUS

(71) Applicant: ROQUETTE FRERES, Lestrem (FR)

(72) Inventor: GABRIEL MACQUART, MONT BERNANCHON (FR)

(21) Appl. No.: 14/779,359

(22) PCT Filed: Mar. 27, 2014
(86) PCT No.: PCT/EP2014/056122

§ 371 Date: Sep. 23, 2015

(30) Foreign Application Priority Data
Mar. 29, 2013 (FR) 1352856

(51) Int. Cl. C12P 23/00; C12P 19/42; C12P 17/16; C12P 17/18

(52) U.S. Cl. CPC C12P 23/00; C12P 17/182; C12P 19/42; C12P 17/165

(57) ABSTRACT

The invention relates to a method for stabilising a biomass of microalgae containing oxidation-sensitive metabolites selected from the group consisting of carotenoids (lutein, etc.), monounsaturated and polyunsaturated fatty acids (palmitoleic acid, oleic acid, linoleic acid, etc.), chlorophyll pigments (chlorophyll A and B, etc.) and vitamins (vitamin B9 and B12, etc.) taken individually or together, more specifically carotenoids, said method comprising the fermentation of said biomass in heterotrophic conditions.

The present invention relates to a method for stabilizing oxidation-sensitive metabolites produced by microalgae, more particularly of the genus Chlorella.

The best-known oxidation-sensitive metabolites include for example the carotenoids.

The carotenoids are pigments, generally orange or yellow, widely occurring in a great many living organisms. Being liposoluble, in general they are easily assimilated by organisms.

They belong to the chemical class of terpenoids, formed from the polymerization of isoprene units with aliphatic or alicyclic structure. It is generally assumed that they follow metabolic pathways similar to those of the lipids.

They are synthesized by all green plants and by many fungi, bacteria (including cyanobacteria) and by all microalgae.

They are absorbed by animals in their food.

The carotenoids have been studied for the prevention of cancer and other human diseases, as they have remarkable antioxidant properties.

Moreover, it is their antioxidant properties that make the carotenoids particularly sensitive to oxidation, as it is well known by a person skilled in the art that an antioxidant substance must itself be easily oxidizable to perform this role.

Representative examples of carotenoids comprise α-carotene, β-carotene and lycopene.

Lycopene and β-carotene are generally present in a free, uncombined form, which is contained within the chloroplasts of plant cells.

The xanthophylls are molecules of a yellow color derived from carotenes by addition of oxygen atoms (alcohol, ketone, epoxy and other functions). They belong to the carotenoid class.

Xanthophylls are abundant in a certain number of yellow or orange fruits and vegetables such as peaches, mangoes, papaya, plums, squashes and oranges.

They also occur in the chloroplasts or chromoplasts of plant cells, notably in the petals of certain flowers colored yellow, orange or red, and in algae, for example brown algae (Phaeophyceae), where they mask chlorophyll.

The xanthophylls are antioxidants that contribute among other things to the health of the eyes.

Examples of xanthophylls comprise lutein, astaxanthin, canthaxanthin, zeaxanthin, cryptoxanthin etc.

The free form of the carotenoids allows better absorption when they are consumed in foodstuffs or as a food supplement.

Lutein is a xanthophyll pigment of formula 4[18-(4-hydroxy-2,6,6-trimethylcyclohex-1-en-1-yl)-3,7,12,16-tetramethyloctadeca-1,3,5,7,9,11,13,15,17-nonaen-1-yl]-3,5,5-trimethylcyclohex-2-en-1-ol found in high concentrations in the macula of the eye and in the central part of the retina.

There, it has an important role in filtering the ultraviolet wavelengths of light to avoid damage to the lens of the eye and the macula.

Moreover, lutein has antioxidant properties that also provide protection of the macula, which is rich in polyunsaturated fatty acids, from light-induced free radicals.

Lutein cannot be produced by the body and consequently must be supplied in the diet.

Thus, lutein is being used more and more in food supplements for preventing and/or treating loss of vision due to age-related macular degeneration (or ARMD), cataracts, and retinitis pigmentosa.

Microalgae of the type Muriellopsis sp., Scenedesmus almeriensis, Chlorella zofingiensis, Chlorella sorokiniana and Chlorella protothecoides have already been proposed as potential sources of lutein.

From the regulatory standpoint, lutein is obtained by solvent extraction from edible strains of fruits and vegetables as well as grasses, alfalfa and Tagetes erecta.

Various amounts of carotenes may also be present.

Lutein may contain fatty substances and waxes naturally present in the original plant material.

Only the following solvents are permitted for extracting them: methanol, ethanol, propanol-2, hexane, acetone, methyl ethyl ketone and carbon dioxide.

Certain commercial preparations of lutein are sold as having “5% or 10% of lutein”. These preparations are in fact purified lutein (esterified or free) added to an inert stabilizer in a proportion from 5 to 15% to stabilize it.

As it is sensitive to light and to oxidation, it must be stored in a sealed container, resistant to light and to oxygen, in a cool, dry place. Despite these precautions, the stability of these compounds is not guaranteed.

Moreover, these storage and handling conditions are by no means easy.

Lutein occurs in a high proportion in certain microalgae such as Chlorella; it is therefore preferable to select this microbial source in order to develop production processes for obtaining larger amounts of lutein profitably, and make up for the losses inherent in the fragility of such molecules.

Moreover, the microalgae of the genus Chlorella contain a unique range of components that includes, besides the aforementioned carotenoids, all the essential amino acids, saturated and unsaturated fatty acids, a large amount of vitamins, minerals and trace elements, as well as valuable components, including chlorophyll, in high concentration.

The microalgae are also an attractive potential food source rich in proteins and other essential nutrients; once dry, conventionally they represent supply of about 45% of proteins, 20% of fatty substances, 20% of carbohydrates, 5% of fibers and 10% of minerals and vitamins.

However, just like the carotenoids, the unsaturated fatty acids have even higher sensitivity to oxidation as their degree of unsaturation increases; thus, polyunsaturated linoleic acid is more sensitive to oxidation than the monounsaturated acids of the oleic and palmitoleic type.

The same applies to the chlorophyll pigments, whose sensitivity to photo-oxidation is familiar to a person skilled in the art.

Thus, it is known that the soluble salts of chlorophyll have antioxidant activity more than 1000 times greater than that of the xanthines, and 20 times greater than that of resveratrol (a polyphenol of the stilbene class present in certain fruits such as grapes, mulberries, etc.).

Chlorophyll supplements are thus marketed in liquid form, as tablets or as capsules. Chlorophyll is often included in green food formulas in powder form.

As for the vitamins, the hydrosoluble B vitamins (vitamin B9 or vitamin B12, for example), are naturally fragile (sensitivity to light, heat and oxidation).

The importance of vitamins A, C and E as antioxidants in the biochemistry of living organisms is also well documented.

To increase the resistance of all these nutrients it is therefore necessary to protect them from “external aggressive factors”, i.e. light and oxygen.

Now, the classical routes for preparing these compounds go via their extraction/purification from their basic biological medium, and then enclosing them in sealed containers.

Antioxidant additives and storage conditions under inert atmosphere are therefore recommended. However, besides the fact that these technologies are complex and expensive to implement, they are not very effective, nor are they completely satisfactory from the standpoint of nutrition and health.

An alternative technical solution has been presented, notably in US patent 2005/0186298, consisting of stabilizing carotenoids in the biomass of the microalgae that produce them.

In this case it is more particularly a matter of stabilizing the biomass of Haemotococcus pluvialis, which produces astaxanthin.

However, this technical solution recommends:
producing the dry biomass (carried out conventionally in phototrophic conditions—growth in the presence of light and CO₂—but may be also produced in heterotrophy—fermentation in the dark in the presence of an assimilable carbon-containing source), and then
combining this biomass with a mixture of at least two antioxidants of the tocopherol type.

Consequently, the underlying problem of the present invention is to propose an alternative method for stabilizing oxidation-sensitive metabolites, and more particularly those produced by microalgae, notably those of the genus Chlorella, by a simple method, without the need to add chemicals such as antioxidants or stabilizers.

With a view to developing a method that is more effective than those known in the prior art, the applicant undertook their own research and succeeded in adapting the technologies for production of microalgae in heterotrophy to achieve this aim.

Culturing of chlorophytes, and more particularly of Chlorella, in heterotrophic conditions is known in a general way for preparing biomasses rich in metabolites of interest, including lutein.

Thus, it has been assumed since the 1960s that it is even possible to obtain much higher yields of pigments than with the same microalgae cultured more conventionally in illuminated auxotrophic conditions.

The choice of this heterotrophic production route, as noted above, in particular has the aim of producing the highest possible lutein contents, thus compensating for their loss by oxidative degradation.

In their article published in 2007 in the review World J. Microbiol. Biotechnol., Vol. 23, pp 1233-1238, Wu et al. thus modeled the production of lutein by heterotrophic fermentation culture in batch mode and fed-batch mode in order to optimize the production of lutein.

However, as far as the applicant knows, none of the documents of the prior art describes or suggests using the biomass itself, produced in heterotrophic conditions, as a vector for stabilizing the metabolites of interest.

Quite on the contrary, it is recommended to add antioxidants to achieve this result.

The present invention therefore relates to a method for stabilizing or for storing oxidation-sensitive metabolites selected from the group consisting of the carotenoids such as lutein, monounsaturated and polyunsaturated fatty acids such as palmitoleic acid, oleic acid or linoleic acid, chlorophyll pigments such as chlorophyll A and B, and vitamins such as vitamins B9 and B12, taken alone or in combination, more particularly carotenoids, said method comprising:
fermenting a biomass of microalgae in heterotrophic conditions comprising a culture phase deficient in a nutrient factor; and
storing the dry biomass in which the oxidation-sensitive metabolites are stabilized.

“Stabilizing the metabolites” means guaranteeing the quality of the metabolites of interest after storing for a period of more than one year, which is notably reflected in protection of the metabolites against oxidative degradation. Notably, storage may be carried out at room temperature under a non-inert atmosphere. The storage step lasts at least 12, 18 or 24 months, preferably at room temperature.

“Oxidation-sensitive metabolites of interest” produced by microalgae of the genus Chlorella means compounds selected from the group consisting of the carotenoids including lutein, monounsaturated and polyunsaturated fatty acids including palmitoleic acid, oleic acid and linoleic acid, chlorophyll pigments such as chlorophyll A and B, and vitamins, including vitamin B9 and notably vitamin B12, more particularly carotenoids.

Culture phase “deficient in a nutrient factor” means a culture phase in which at least one of the nutrient factors of the microalga is supplied in an amount that is insufficient to allow its normal growth. It should be noted that “insufficient amount” is not understood as zero supply of this nutrient factor. This nutrient-deficient phase then results in slowing (limiting) cellular metabolism, without inhibiting it completely.

Culture is for example carried out in conditions such that one of the nutrient factors is supplied to the medium at a rate below the rate of consumption that the microalga could achieve without limitation.

This is also reflected in absence of residual nutrient factor in the culture medium, as the microalga consumes this nutrient factor as it is supplied.

Preferably, the nutrient factor is the carbon-containing source, and more particularly glucose.

The present invention is thus particularly suitable for stabilizing metabolites such as the carotenoids, whose extreme sensitivity to oxidation is familiar to a person skilled in the art.

In the more particular context of the invention, stabilization of the carotenoids thus also guarantees stabilization of the other metabolites produced by the microalgae, said metabolites being susceptible to degradation by oxidation.

More particularly, the oxidation-sensitive metabolites are stored in the cells of the microalgae contained in the biomass.

The method according to the present invention makes it unnecessary to add exogenous antioxidant or stabilizer for preserving the oxidation-sensitive metabolites. Thus, preferably, the method does not comprise adding exogenous antioxidant or stabilizer to said dry biomass.

The invention thus provides a natural biomass with a guaranteed content of metabolites of interest, such as pigments.

Here, biomass of microalgae means microalgae, more particularly of the genus Chlorella, such as Chlorella sorokiniana. In a very particular embodiment, the strain of Chlorella sorokiniana is the strain UTEX 1663—The Culture Collection of Algae at the University of Texas at Austin—USA.

More particularly, the metabolites of interest to be stabilized, as in the examples given here, will be total carotenoids, including lutein, chlorophylls and vitamins. In a preferred embodiment, the metabolite of interest to be stabilized is lutein.

Vitamin B12, which is not produced naturally by the microalga when cultured in heterotrophy, is added to the culture medium and assimilated by the latter.

In a preferred embodiment of the method according to the invention, fermentation is carried out in particular conditions of heterotrophic culture that guarantee optimal efficiency with respect to stabilization of the oxidative degradation-sensitive metabolites of interest produced by the microalgae.

Thus, the method comprises fermenting a biomass of microalgae in heterotrophic conditions with a first step of growth of the biomass and with a second step of culture deficient in a nutrient factor.

Production of biomass thus comprises:
a first step of fermentation in batch mode,
a second step of fermentation in fed-batch mode which, when the carbon-containing source is completely consumed by the microalga, involves continuous supply of said carbon-containing source at a rate below the rate of consumption that the microalga could achieve without limitation.

The applicant recommends carrying out, prior to the step of production of the biomass proper, a first step of preculture (by fermentation in batch mode) which then allows production of an amount of biomass of microalgae necessary for seeding the production fermenters proper.

For example, if we decide to culture a strain of Chlorella such as Chlorella sorokiniana, after this first step of preculture we obtain a cell density (measured conventionally by the optical density at 600 nm) with a value between 50 and 60, as will be shown in the examples given below.

Regarding production of the biomass of microalgae, it therefore comprises a first step of fermentation in batch mode, with a culture medium for example identical to that employed in the preculture step.

When there is complete consumption of the carbon-containing source by the microalga (here: residual glucose in the culture medium=0 g/l), said carbon-containing source is added continuously at a rate lower than its rate of consumption by the microalga. In a preferred embodiment, the deficient nutrient factor is glucose.

The applicant has thus overcome a technical presumption, since it is commonly assumed that to optimize the production of lutein by Chlorella (article by Wu et al. cited above), if the high glucose concentration inhibits growth and the production of lutein, it is recommended to give preference to a minimum glucose concentration, between 5 and 24 g/l in the case of production of lutein by C. pyrenoidosa.

Therefore the culture conditions recommended according to the invention are not compatible with those commonly assumed in the prior art for optimizing lutein production.

In the case of Chlorella sorokiniana, the applicant recommends adding glucose at a rate above 1 g/l/h, maintaining residual glucose at 0 g/l. For example, the rate of glucose supply may be between 1 and 5 g/l/h, more particularly between 2 and 4 g/l/h. This rate is selected in such a way that the residual glucose in the culture medium is 0 g/l. This rate may be defined on the basis of the rate of consumption of glucose in the absence of limitation. Thus, the rate of glucose supply may be 90, 80, 70, 60 or 50% of this rate of consumption of glucose in the absence of limitation.

This rate is thus positioned at about 2 g/l/h at the start of fed-batch operation, and it can increase to 4 g/l/h at the end of culture.

The rate of supply of the deficient nutrient factor is preferably selected so as to decrease or slow the cell growth rate, while maintaining growth at a non-zero rate. Notably, it is proposed to lower the growth rate by 10 to 60% relative to the growth rate without glucose limitation, notably 10, 15, 20, 25, 30, 35, 40, 45, 50 or 55% relative to the growth rate without glucose limitation. Preferably, the growth rate is reduced by 15 to 55%.

For example, for the strain of Chlorella sorokiniana, the applicant recommends selecting a rate of glucose supply allowing growth μ of at least 0.04 ⁻¹, for example between 0.06 h⁻¹ and 0.09 h⁻¹.

Thus, the rate of supply of nutrient factor is such that it allows cell growth μ of at least 0.04 h⁻¹, for example between 0.06 h⁻1 and 0.09 h⁻¹.

The duration of the culture phase deficient in a nutrient factor, notably glucose, is at least 1 h, preferably at least 10 h, more preferably at least 20 h, and notably between 30 and 60 h.

Fermentation is stopped (supply of glucose stopped) when the desired amount of biomass is reached (for example between 30 and 80 g/l).

A stability study (for 14 months and 23 months) was carried out for the purpose of studying the evolution of the content of carotenoids (lutein), vitamins (B9 and more particularly B12) and total fatty acids in the biomass obtained by fermentation in heterotrophic conditions, in comparison with a biomass produced in phototrophic conditions.

As will be shown in the examples, there is far better stability of the metabolites produced by Chlorella when the biomass is prepared in heterotrophic conditions according to the preferred method of the invention.

Thus, for example, the biomass obtained in a photobioreactor had lost practically 80% of its lutein concentration after 14 months of storage, whereas that of the biomass prepared in heterotrophic conditions was unchanged.

The present invention also relates to the use of a biomass of microalgae produced in heterotrophic conditions comprising a culture phase deficient in a nutrient factor for stabilizing their oxidation-sensitive metabolites. Preferably, the microalgae are selected from the group of microalgae of the genus Chlorella, more particularly Chlorella sorokiniana. Preferably, the limiting nutrient factor is the carbon-containing source, in particular glucose.

The present invention also relates to a biomass of microalgae of the genus Chlorella, more particularly Chlorella sorokiniana, obtained by the method according to the present invention, characterized in that it contains at least 1 g of lutein per kg of biomass after storage for at least 12, 18 or 24 months at room temperature without adding exogenous antioxidant or stabilizer to said dry biomass.

The invention will be better understood from the examples given below, which are intended to be illustrative and nonlimiting.

### EXAMPLES

### Example 1

### Preparation of the Biomass of C. Sorokiniana Cultured in Heterotrophic Conditions According to the Preferred Embodiment of the Invention

Phase 1. Preculture

Preculture allows reactivation of the strains and inoculation of the production fermenter.

It is carried out in conical flasks starting from a frozen tube of a strain of Chlorella sorokiniana (strain UTEX 1663—The Culture Collection of Algae at the University of Texas at Austin—USA) and with 600 ml of medium of the composition as presented in Table 1 below:

TABLE 1

| | | |
|---|---|---|
| Macro | Glucose | 20 |
| elements | K₂HPO₄•3H₂O | 0.7 |
| (g/L) | MgSO₄•7H₂O | 0.34 |
| | Citric acid | 1.0 |
| | Urea | 1.08 |
| | Na₂SO₄ | 0.2 |
| | Na₂CO₃ | 0.1 |
| | Yeast extract | 1 |
| | clerol FBA 3107 (antifoaming agent) | 0.5 |
| Micro | Na₂EDTA | 10 |
| elements | CaCl₂•2H₂O | 80 |
| (mg/L) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

The pH is adjusted to 7 before sterilization by adding 8N NaOH.

Incubation takes place at 28° C.±1° C. with stirring at 110 rpm (INFORS Multitron stirrer) for 72 hours.

The final concentration of biomass obtained at the end of incubation of each conical flask, from measurement of OD at 600 nm, is positioned at about 50-60.

Phase 2. Production

The parameters for carrying out fermentation are presented in Table 2 below.

TABLE 2

| | |
|---|---|
| Volume | 13.5 L after Inoculation - 16 to 20 L finally |
| Temperature | 28-30° C. |
| pH | 6.5-6.6 with NH₃ 28% w/w |
| pO₂ | >20% (maintained by stirring) |
| Stirring | 300 rpm |
| Air flow rate | 15 L/min |

Step 1: Fermentation in Batch Mode

The medium for batch mode is identical to that for preculture (Table 1), but without yeast extract, and urea is replaced with NH₄Cl (Table 3 below).

TABLE 3

| | | |
|---|---|---|
| Macro | Glucose | 20 |
| elements | K₂HPO₄•3H₂O | 0.7 |
| (g/L) | MgSO₄•7H₂O | 0.34 |
| | Citric acid | 1.0 |
| | NH₄Cl | 1.88 |
| | Na₂SO₄ | 0.2 |
| | clerol FBA 3107 (antifoaming agent) | 0.5 |
| Micro | Na₂EDTA | 10 |
| elements | CaCl₂ | 80 |
| (mg/L) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

Step 2: Fermentation in Fed-Batch Mode

Vitamin B12 is also added in the batch phase, at a rate of 0.47 μg/l of medium, so that it is stored in the biomass at a value of the order of 400 μg/100 g of dry biomass.

In the fed-batch phase, supply of complete medium is provided with continuous feed of glucose at a rate below the rate of consumption permitted by the strain so that the residual content in the medium is zero.

This rate of glucose supply is at about 2 g/L_{AI}/h (AI=after inoculation) at the start of “fed-batch” and it may increase to 4 g/L_{AI}/h at the end of culture. Growth μ during this step is between 0.07 and 0.08 h⁻¹.

The glucose concentration in the feed solution may range from 400 to 800 g/L.

The salts are supplied continuously, separately or mixed with the glucose.

The first addition must be made as soon as the batch has ended, but the salts must not be supplied just once, to avoid inhibiting the growth of the strain.

Table 4 below gives the salt requirements per 100 g of glucose:

TABLE 4

| | | |
|---|---|---|
| Macro | Glucose | 100 |
| elements | K₂HPO₄•3H₂O | 6.75 |
| (g) | MgSO₄•7H₂O | 1.7 |
| | Citric acid | 5.0 |
| | Na₂SO₄ | 1.0 |
| Micro | Na₂EDTA | 50 |
| elements | CaCl₂•2H₂O | 400 |
| (mg) | FeSO₄•7H₂O | 200 |
| | MnSO₄•4H₂O | 2.1 |
| | CoSO₄•7H₂O | 1.2 |
| | CuSO₄•5H₂O | 1.2 |
| | ZnSO₄•7H₂O | 2.5 |
| | H₃BO₃ | 0.6 |
| | (NH₄)₆Mo₇O₂₇•4H₂O | 0.2 |

Feed of glucose is stopped when the cell density reaches 80 g/l. This stops culture.

The biomass is atomized to dry matter >95%.

### Example 2

### Studies of Room Temperature Stability of the Oxidation-Sensitive Metabolites Produced by Chlorella sorokiniana

A stability study was carried out for a period of 14 and 23 months at room temperature, for the purpose of studying the evolution of the content of carotenoids, lutein, vitamin B9 and more particularly vitamin B12, extracted from the biomasses obtained in heterotrophy (by fermentation) or in autotrophy (photobioreactors).

This study demonstrates the effect of the method of culture of Chlorella on the stability of these oxidation-sensitive metabolites.

Two biomasses are compared:
the first produced according to the conditions of example 1,
the second produced in a photobioreactor (such as PBR 4000), in conventional conditions (Pulz et al., 2000, in Rehm H.-J., Reed G. (Eds), Biotechnology, Vol. 10, Second edition, Weinheim, 105-136).

The measurements are performed on 100 g of biomass at more than 95% DM (dry matter), produced in these two operating conditions.

Evolution of the Content of Total Carotenoids and of Lutein (Determinations Performed by HPLC)

| | Biomass produced by | | | Biomass produced in a | | |
|---|---|---|---|---|---|---|
| | fermentation | | | photobioreactor | | |
| | | t = 14 | t = 23 | | t = 14 | t = 23 |
| | t = 0 | months | months | t = 0 | months | months |
| Lutein | 2100 | 1950 | 1475 | 2650 | 525 | 375 |
| (mg/kg) | | | | | | |
| Total | 0.49 | 0.32 | 0.27 | 0.51 | 0.17 | 0.14 |
| carotenoids | | | | | | |
| (g/100 g) | | | | | | |

The stability of the total carotenoids is higher in the biomass obtained in heterotrophy.

The same trend is observed for lutein; the fermented biomass provides better protection of the lutein against degradation.

Evolution of the Content of Vitamins (Assays Performed According to Methods AOAC 952.20—Vitamin B12)

Supplied by the culture medium in heterotrophy, vitamin B12 was well assimilated by the biomass of Chlorella sorokiniana; in this case its content is of the order of 363 μg per 100 g of dry biomass.

| | Biomass produced by | | | Biomass produced in a | | |
|---|---|---|---|---|---|---|
| | fermentation | | | photobioreactor | | |
| | | t = 14 | t = 23 | | t = 14 | t = 23 |
| | t = 0 | months | months | t = 0 | months | months |
| Vitamin B12 | 363 | 304 | nd | 120 | 107 | nd |
| (μg/100 g) | | | | | | |

nd denotes not determined.

Stability of vitamin B12 is observed throughout the study in both of these cases. The values varied around 300 μg/100 g for the biomass produced in heterotrophy and around 105 μg/1400 g for the biomass produced in autotrophy.

Evolution of the Content of Chlorophylls (Determined by Spectrophotometry)

| | Biomass produced by | | | Biomass produced in a | | |
|---|---|---|---|---|---|---|
| | fermentation | | | photobioreactor | | |
| | | t = 14 | t = 23 | | t = 14 | t = 23 |
| | t = 0 | months | months | t = 0 | months | months |
| Chlorophyll A | 2.21 | 1.7 | 1.48 | 2.33 | 1.32 | 1.07 |
| (g/100 g) | | | | | | |
| Chlorophyll B | 0.55 | 0.51 | 0.5 | 0.57 | 0.57 | 0.53 |
| (g/100 g) | | | | | | |

The content of chlorophyll B remains stable in both operating conditions, but this is not the case for the content of chlorophyll A, which is stabilized far better in the biomass produced by fermentation.

In conclusion, the metabolites produced by the microalgae are more stable in the biomass produced by fermentation (which reflects their better protection against oxidative degradation, to which they are sensitive).

This phenomenon is even more pronounced for the lutein content, which is remarkably more stable in the fermented biomass (whereas it is more than 80% degraded in the biomass produced in autotrophy).

1-13. (canceled)

14. A method for stabilizing or for storing oxidation-sensitive metabolites selected from the group consisting of the carotenoids, monounsaturated and polyunsaturated fatty acids, chlorophyll pigments, and vitamins, alone or in combination comprising:
fermenting a biomass of microalgae in heterotrophic conditions comprising a culture phase deficient in a nutrient factor; and
storing the dry biomass in which the oxidation-sensitive metabolites are stabilized.

15. The method of claim 14, wherein the microalgae are of the genus Chlorella.

16. The method of claim 15, wherein the microalgae are Chlorella sorokiniana.

17. The method of claim 15, wherein the method does not comprise adding exogenous antioxidant or stabilizer to said dry biomass.

18. The method of claim 15, wherein the deficient nutrient factor is the carbon-containing source.

19. The method of claim 18, wherein the fermenting of said biomass of microalgae in heterotrophic conditions comprises:
a first step of fermentation in batch mode,
a second step of fermentation in fed-batch mode which, when the carbon-containing source is completely consumed by the microalgae, involves continuous supply of said carbon-containing source at a rate lower than its rate of consumption by the microalgae.

20. The method of claim 18, wherein the deficient nutrient factor is glucose and in that it is supplied to the culture at a rate above 1 g/l/h.

21. The method of claim 19, wherein the deficient nutrient factor is glucose and in that it is supplied to the culture at a rate between 1 and 5 g/l/h.

22. The method of claim 15, wherein that the duration of the deficient culture phase is at least 1 h.

23. The method of claim 22, wherein that the duration of the deficient culture phase at least 10 h.

24. The method of claim 22, wherein that the duration of the deficient culture phase is between 30 and 60 h.

25. The method of claim 15, wherein the storage step lasts at least 12, 18 or 24 months at room temperature.

26. The method of claim 15, wherein the oxidation-sensitive metabolite is lutein.

27. A biomass of microalgae of the genus Chlorella, obtained by the method of claim 15, wherein said biomass contains at least 1 g of lutein per kg of biomass after storage for at least 12, 18 or 24 months at room temperature without adding exogenous antioxidant or stabilizer to said dry biomass.
